(19) United States
(12) Patent Application Publication
(10) Pub. No.: US 2022/0267341 A1
(43) Pub. Date: Aug. 25, 2022

(54) SOLID FORMS OF TERT-BUTYL (S)-2((2S,3R)-1-AMINO-3-HYDROXY-1-OXOBU TAN-2-YL)-1-OXO-2, 5-DIAZASPIRO [3.4] OCTAN E-5-CARBOXYLATE AND METHODS OF PREPARING THEM

(71) Applicant: NAUREX INC., Madison, NJ (US)

(72) Inventors: Xiaoda YUAN, Costa Mesa, CA (US); Shaoxin FENG, Tustin, CA (US); Danny T. DING, Costa Mesa, CA (US); William R. PERRAULT, Kalamazoo, MI (US)

(21) Appl. No.: 17/621,907

(22) PCT Filed: Jun. 23, 2020
(86) PCT No.: PCT/US2020/039163

§ 371 Date: Dec. 22, 2021

Related U.S. Application Data
(60) Provisional application No. 62/865,826, filed on Jun. 24, 2019.

(51) Int. Cl. C07D 487/10

(52) U.S. Cl. CPC C07D 487/10; C07B 2200/13

(57) ABSTRACT

Solid state forms of tert-buty 1 (S)-2-((2S,3R)-1-amino-3-hydroxy-1-oxobutan-2-yl)-1-oxo-2,5-diazaspiro[3.4] octane-5-carboxylate, pharmaceutical compositions, preparation, and uses thereof.

### CROSS-REFERENCE TO RELATED APPLICATIONS

This application claims priority to and/or the benefit of United States provisional application U.S. 62/865,826 filed Jun. 24, 2019 which is hereby incorporated by reference in its entirety and serves as the basis of a priority and/or benefit claim for the present application.

### TECHNICAL FIELD

The subject matter described herein relates to solid state forms, for example, crystalline forms and amorphous forms, of tert-butyl (S)-2-((2S,3R)-1-amino-3-hydroxy-1-oxobutan-2-yl)-1-oxo-2,5-diazaspiro[3.4]octane-5-carboxylate, pharmaceutical compositions thereof, methods for preparation and uses thereof.

### BACKGROUND

N-methyl-D-aspartate receptor (NMDA receptor) is believed to play a major role in the synaptic plasticity that underlies many higher cognitive functions, such as memory acquisition, retention and learning, as well as in certain cognitive pathways and in the perception of pain. The NMDA receptor also appears to be involved in a broad spectrum of CNS disorders. NMDA receptor modulators therefore can provide pharmaceutical benefits.

Tert-butyl (S)-2-((2S,3R)-1-amino-3-hydroxy-1-oxobutan-2-yl)-1-oxo-2,5-diazaspiro[3.4]octane-5-carboxylate is disclosed in U.S. Pat. No. 9,512,134 (said patent is incorporated herein by reference in its entirety) as a NMDA receptor modulators that can be useful for treating, for example, depression. There remains a need for stable solid state forms of Compound A that can be used in pharmaceutical compositions and their manufacture.

### BRIEF SUMMARY

The following aspects and embodiments thereof described and illustrated below are meant to be exemplary and illustrative, not limiting in scope.

In one aspect, solid forms of tert-butyl (S)-2-((2S,3R)-1-amino-3-hydroxy-1-oxobutan-2-yl)-1-oxo-2,5-diazaspiro[3.4]octane-5-carboxylate (hereinafter “Compound A”) are provided. Compound A has the structure below:

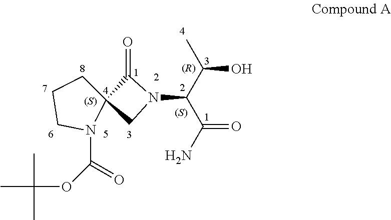

In some embodiments, disclosed herein is a crystalline anhydrous form of Compound A designated as crystalline Form I of Compound A.

In some embodiments, disclosed herein is a crystalline dihydrate form of Compound A designated as crystalline Form II of Compound A.

In some embodiments, disclosed herein is an amorphous form of Compound A.

In another aspect, disclosed herein is a pharmaceutical composition, comprising at least one pharmaceutically acceptable carrier and a solid form of Compound A.

In some embodiments, disclosed herein is a pharmaceutical composition, comprising at least one pharmaceutically acceptable excipient and crystalline Form I of Compound A.

In some embodiments, disclosed herein is a pharmaceutical composition, comprising at least one pharmaceutically acceptable excipient and crystalline Form II of Compound A.

In some embodiments, disclosed herein is a pharmaceutical composition, comprising a pharmaceutically acceptable excipient and amorphous form of Compound A.

In another aspect, disclosed is a method of treating a subject in recognized need of treatment for a disease or disorder responsive to NMDA modulation, such as major depressive disorder, comprising administering to said subject in need thereof a therapeutically effective amount of a pharmaceutical composition, wherein the pharmaceutical composition comprises a pharmaceutically acceptable excipient and a solid form of Compound A.

In some embodiments, disclosed is a method of treating a subject in recognized need of treatment for a disease or disorder responsive to NMDA modulation, such as major depressive disorder, comprising administering to said subject in need thereof a therapeutically effective amount of a pharmaceutical composition, wherein the pharmaceutical composition comprises a pharmaceutically acceptable excipient and crystalline Form I of Compound A.

In some embodiments, disclosed is a method of treating a subject in recognized need of treatment for a disease or disorder responsive to NMDA modulation, such as major depressive disorder, comprising administering to said subject in need thereof a therapeutically effective amount of a pharmaceutical composition, wherein the pharmaceutical composition comprises a pharmaceutically acceptable excipient and crystalline Form II of Compound A.

In some embodiments, disclosed is a method of treating a subject in recognized need of treatment for a disease or disorder responsive to NMDA modulation, such as major depressive disorder, comprising administering to said subject in need thereof a therapeutically effective amount of a pharmaceutical composition, wherein the pharmaceutical composition comprises a pharmaceutically acceptable excipient and amorphous form of Compound A.

In another aspect, disclosed is a method of preparing a solid form of Compound A.

In some embodiments, disclosed is a method of preparing crystalline Form I of Compound A.

In some embodiments, disclosed is a method of preparing crystalline Form II of Compound A.

In some embodiments, disclosed is a method of preparing amorphous form of Compound A.

Some non-liming exemplary embodiments are listed below.

Example embodiment 1: A method of preparing the solid crystalline Form I of compound A:

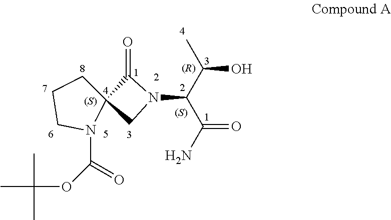

the method comprising:
dissolving compound A in ethyl acetate and heating the solution;
cooling the solution; and
adding a diisopropyl ether to the solution.

Example embodiment 2: The method of example embodiment 1, wherein the solution is heated to between about 65° C. and about 70° C.

Example embodiment 3: The method of example embodiment 1, wherein the solution is cooled to about 25° C.

Example embodiment 4: The method of any one of example embodiments 1 to 3, wherein the solid crystalline Form I of compound A has peaks (2θ) chosen from those having about the following values: 6.9, 8.4, 10.3, and 12.8 in a powder X-ray diffraction pattern.

Example embodiment 5: The method of example embodiment 4, wherein the solid crystalline Form I of compound A further has one or more peaks (2θ) chosen from those having about the following values: 13.7, 15.3, 15.7, 16.8, 17.3, 18.5, and 19.9 in a powder X-ray diffraction pattern.

Example embodiment 6: The of the method of any one of example embodiments 1 to 3, wherein the solid crystalline Form I of compound A has peaks (2θ) chosen from those having about the following values: 6.9, 8.4, 10.3, 12.8, and 13.7 in a powder X-ray diffraction pattern.

Example embodiment 7: The method on any one of example embodiments 1 to 3, wherein the solid crystalline Form I of compound A has peaks (2θ) chosen from those having about the following values 6.9, 8.4, 10.3, 12.8, 13.7, 15.3, and 15.7 in a powder X-ray diffraction pattern.

Example embodiment 8: The method of any one of example embodiments 1 to 3, having peaks (2θ) chosen from those having about the following values 6.9, 8.4, 10.3, 12.8, 13.7, 15.3, 15.7, and 16.8 in a powder X-ray diffraction pattern.

Example embodiment 9: The method of any one of example embodiments 1 to 3, wherein the solid crystalline Form I of compound A has peaks (2θ) chosen from those having about the following values 6.9, 8.4, 10.3, 12.8, 13.7, 15.3, 15.7, 16.8, and 17.3 in a powder X-ray diffraction pattern.

Example embodiment 10: The method of any one of example embodiments 1 to 3, wherein the solid crystalline Form I of compound A has peaks (2θ) chosen from those having about the following values 6.9, 8.4, 10.3, 12.8, 13.7, 15.3, 15.7, 16.8, 17.3, and 18.5 in a powder X-ray diffraction pattern.

Example embodiment 11: The method of any one of example embodiments 1 to 3, wherein the solid crystalline Form I of compound A has 3, 4 or 5 peaks (2θ) chosen from those having about the following values 6.9, 8.4, 10.3, 12.8, 13.7, 15.3, 15.7, 16.8, 17.3, 18.5, and 19.9 in a powder X-ray diffraction patterns.

Example embodiment 12: The method of any one of example embodiments 1 to 3, wherein the solid crystalline Form I of compound A has an XRPD pattern substantially similar to one of the XRPD patterns shown in FIG. 1.

Example embodiment 13: The method of any one of example embodiments 1 to 12, wherein the solid crystalline Form I of compound A has a DSC with endothermic peaks at about 159° C.

Example embodiment 14: A solid crystalline form of compound A:

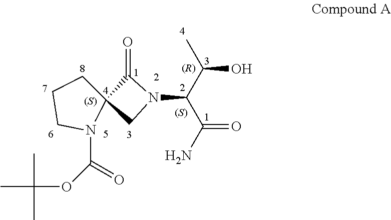

wherein the solid crystalline form is crystalline Form I of Compound A.

Example embodiment 15: The solid crystalline form of example embodiment 14, having peaks (2θ) chosen from those having about the following values: 6.9, 8.4, 10.3, and 12.8 in a powder X-ray diffraction pattern.

Example embodiment 16: The solid crystalline form of example embodiment 15, further having one or more peaks (2θ) chosen from those having about the following values: 13.7, 15.3, 15.7, 16.8, 17.3, 18.5, and 19.9 in a powder X-ray diffraction pattern.

Example embodiment 17: The solid crystalline form of example embodiment 14, having peaks (2θ) chosen from those having about the following values: 6.9, 8.4, 10.3, 12.8, and 13.7 in a powder X-ray diffraction pattern.

Example embodiment 18: The solid crystalline form of example embodiment 14, having peaks (2θ) chosen from those having about the following values 6.9, 8.4, 10.3, 12.8, 13.7, 15.3, and 15.7 in a powder X-ray diffraction pattern.

Example embodiment 19: The solid crystalline form of example embodiment 14, having peaks (2θ) chosen from those having about the following values 6.9, 8.4, 10.3, 12.8, 13.7, 15.3, 15.7, and 16.8 in a powder X-ray diffraction pattern.

Example embodiment 20: The solid crystalline form of example embodiment 14, having peaks (2θ) chosen from those having about the following values 6.9, 8.4, 10.3, 12.8, 13.7, 15.3, 15.7, 16.8, and 17.3 in a powder X-ray diffraction pattern.

Example embodiment 21: The solid crystalline form of example embodiment 14, having peaks (2θ) chosen from those having about the following values 6.9, 8.4, 10.3, 12.8, 13.7, 15.3, 15.7, 16.8, 17.3, and 18.5 in a powder X-ray diffraction pattern.

Example embodiment 22: The solid crystalline form of example embodiment 14, having 3, 4 or 5 peaks (2θ) chosen from those having about the following values 6.9, 8.4, 10.3, 12.8, 13.7, 15.3, 15.7, 16.8, 17.3, 18.5, and 19.9 in a powder X-ray diffraction patterns.

Example embodiment 23: The solid crystalline form of example embodiment 14, having an XRPD pattern substantially similar to one of the XRPD patterns shown in FIG. 1.

Example embodiment 24: The solid crystalline form of any one of example embodiments 14 to 23, having a DSC with endothermic peaks at about 159° C.

Example embodiment 25: A solid composition comprising the solid crystalline form of any one of example embodiments 14 to 24, wherein the solid composition is at least 99%, at least 95%, at least 90%, at least 80%, at least 70%, at least 60%, or at least 50%, by weight, free of any other solid forms of Compound A.

Example embodiment 26: A pharmaceutical composition comprising the solid crystalline form of any one of example embodiments 14 to 24 and a pharmaceutically acceptable excipient.

Example embodiment 27: The pharmaceutical composition of example embodiment 26, wherein the solid crystalline form is at least 99%, at least 95%, at least 90%, at least 80%, at least 70%, at least 60%, or at least 50%, by weight, of the total amount of tert-butyl (S)-2-((2S,3R)-1-amino-3-hydroxy-1-oxobutan-2-yl)-1-oxo-2,5-diazaspiro[3.4]octane-5-carboxylate in the pharmaceutical composition.

Example embodiment 28: A solid crystalline form of Compound A:

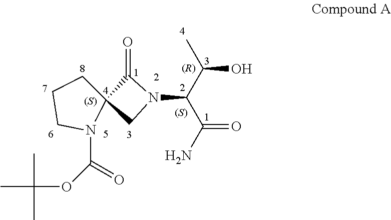

wherein the solid crystalline form is crystalline Form II of Compound A.

Example embodiment 29: The solid crystalline form of example embodiment 28, having peaks (2θ) chosen from those having about the following values: 9.4, 10.8, 11.9, and 13.0 in a powder X-ray diffraction patterns.

Example embodiment 30: The solid crystalline form of example embodiment 29, further having one or more peaks (2θ) chosen from those having about the following values: 13.7, 15.5, 16.0, 20.0, 20.4, 21.3 and 23.3 in a powder X-ray diffraction pattern.

Example embodiment 31: The solid crystalline form of example embodiment 28, having peaks (2θ) chosen from those having about the following values: 9.4, 10.8, 11.9, 13.0, and 13.7 in a powder X-ray diffraction pattern.

Example embodiment 32: The solid crystalline form of example embodiment 28, having peaks (2θ) chosen from those having about the following values 9.4, 10.8, 11.9, 13.0, 13.7, 15.5, and 16.0 in a powder X-ray diffraction pattern.

Example embodiment 33: The solid crystalline form of example embodiment 28, having peaks (2θ) chosen from those having about the following values 9.4, 10.8, 11.9, 13.0, 13.7, 15.5, 16.0, 20.0, and 20.4 in a powder X-ray diffraction pattern.

Example embodiment 34: The solid crystalline form of example embodiment 28, having peaks (2θ) chosen from those having about the following values 9.4, 10.8, 11.9, 13.0, 13.7, 15.5, 16.0, 20.0, 20.4, and 21.3 in a powder X-ray diffraction pattern.

Example embodiment 35: The solid crystalline form of example embodiment 28, having peaks (2θ) chosen from those having about the following values 9.4, 10.8, 11.9, 13.0, 13.7, 15.5, 16.0, 20.0, 20.4, 21.3 and 23.3 in a powder X-ray diffraction pattern.

Example embodiment 36: The solid crystalline form of example embodiment 28, having 3, 4 or 5 peaks (2θ) chosen from those having about the following values 9.4, 10.8, 11.9, 13.0, 13.7, 15.5, 16.0, 20.0, 20.4, 21.3 and 23.3 in a powder X-ray diffraction pattern.

Example embodiment 37: The solid crystalline form of example embodiment 28, having an XRPD pattern substantially similar to one of the two XRPD patterns shown in FIG. 3.

Example embodiment 38: The solid crystalline form of any one of example embodiments 28 to 37, having a DSC with endothermic peaks at about 82° C. and at about 159° C.

Example embodiment 39: The solid crystalline form of any one of example embodiments 28 to 38, having a TGA showing dehydration approximately at above 60° C., with a loss of water of approximately 9.6% by weight.

Example embodiment 40: The solid crystalline form of any one of example embodiments 28 to 39, having a DVS showing about 11% change in mass at 0% RH and 25° C. and the mass does not lose water at or above 20% RH.

Example embodiment 41: A solid composition comprising the solid crystalline form of any one of example embodiments 28 to 40, wherein the solid composition is at least 99%, at least 95%, at least 90%, at least 80%, at least 70%, at least 60%, or at least 50%, by weight, free of any other solid forms of Compound A.

Example embodiment 42: A pharmaceutical composition comprising the solid crystalline form of any one of example embodiments 28 to 40 and a pharmaceutically acceptable excipient.

Example embodiment 43: The pharmaceutical composition of example embodiment 42, wherein the solid crystalline form is at least 99%, at least 95%, at least 90%, at least 80%, at least 70%, at least 60%, or at least 50%, by weight, of the total amount of tert-butyl (S)-2-((2S,3R)-1-amino-3-hydroxy-1-oxobutan-2-yl)-1-oxo-2,5-diazaspiro[3.4]octane-5-carboxylate in the pharmaceutical composition.

Example embodiment 44: A solid amorphous form of Compound A:

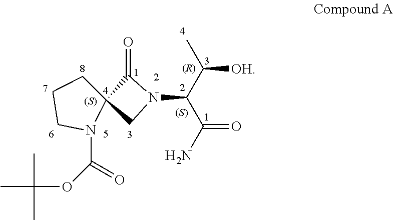

Example embodiment 45: The solid amorphous form of example embodiment 44, having an amorphous halo in a powder X-ray diffraction pattern.

Example embodiment 46: The solid amorphous form of example embodiment 44, having an XRPD pattern that is substantially similar to FIG. 7.

Example embodiment 47: A pharmaceutical composition comprising the amorphous form of any one of example embodiments 44 to 46 and a pharmaceutically acceptable excipient.

Example embodiment 48: The pharmaceutical composition of example embodiment 47, wherein the amorphous form is at least 99%, at least 95%, at least 90%, at least 80%, at least 70%, at least 60%, or at least 50%, by weight, of the total amount of tert-butyl (S)-2-((2S,3R)-1-amino-3-hydroxy-1-oxobutan-2-yl)-1-oxo-2,5-diazaspiro[3.4]octane-5-carboxylate in the pharmaceutical composition.

Example embodiment 49: A method of treating a subject in recognized need of treatment for a disease or disorder responsive to NMDA modulation, comprising administering to said subject in need thereof a therapeutically effective amount of a pharmaceutical composition of any one of example embodiments 26, 27, 42, 43, 47, and 46.

Example embodiment 50: The method of example embodiment 49, wherein the disease or disorder is selected from autism, anxiety, depression, bipolar disorder, attention deficit disorder, attention deficit hyperactivity disorder (ADHD), schizophrenia, a psychotic disorder, a psychotic symptom, social withdrawal, obsessive-compulsive disorder (OCD), phobia, post-traumatic stress syndrome, a behavior disorder, an impulse control disorder, a substance abuse disorder, a sleep disorder, a memory disorder, a learning disorder, urinary incontinence, multiple system atrophy, progressive supra-nuclear palsy, Friedrich's ataxia, Down's syndrome, fragile X syndrome, tuberous sclerosis, olivio-ponto-cerebellar atrophy, cerebral palsy, drug-induced optic neuritis, ischemic retinopathy, diabetic retinopathy, glaucoma, dementia, AIDS dementia, Alzheimer's disease, Huntington's chorea, spasticity, myoclonus, muscle spasm, Tourette's syndrome, epilepsy, cerebral ischemia, stroke, a brain tumor, traumatic brain injury, cardiac arrest, myelopathy, spinal cord injury, peripheral neuropathy, acute neuropathic pain, and chronic neuropathic pain.

Example embodiment 51: The method of example embodiment 50, wherein the substance abuse disorder is selected from a withdrawal symptom, opiate addiction, nicotine addiction, and ethanol addition.

Example embodiment 52: The method of example embodiment 50, wherein the memory disorder is selected from a deficit, loss, and reduced ability to make new memories.

Example embodiment 53: The method of example embodiment 49, wherein the disease or disorder is major depressive disorder.

Example embodiment 54: A crystal form of tert-butyl (S)-2-((2S,3R)-1-amino-3-hydroxy-1-oxobutan-2-yl)-1-oxo-2,5-diazaspiro[3.4]octane-5-carboxylate having an Orthorhombic crystal system, a P2₁2₁2₁ space group, and the following unit cell dimensions: a=5.85088 (9) Å, b=11.57133 (12) Å, and c=25.8340 (3)Å, α=β=γ=90°, V=1749.02 (4) Å³, Z=4.

Example embodiment 55: A crystal form of tert-butyl (S)-2-((2S,3R)-1-amino-3-hydroxy-1-oxobutan-2-yl)-1-oxo-2,5-diazaspiro[3.4]octane-5-carboxylate dihydrate having an Orthorhombic crystal system, a P2₁2₁2₁ space group, and the following unit cell dimensions: a=8.9035 (2) Å, b=10.5404 (2) Å, and c=21.3018 (5)Å, α=β=γ=90°, V=1999.10 (8) Å³, Z=4.

Example embodiment 56: A solid crystalline form of compound A:

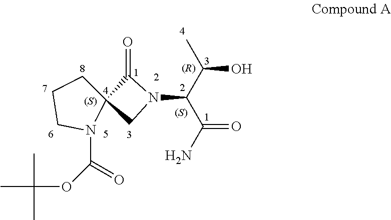

substantially as described herein.

Example embodiment 57: A solid crystalline Form I of compound A:

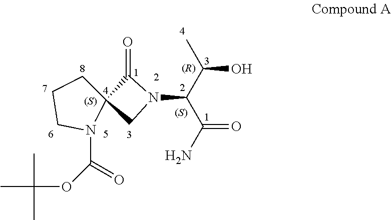

substantially as described herein.

Example embodiment 58: A solid crystalline Form II of compound A:

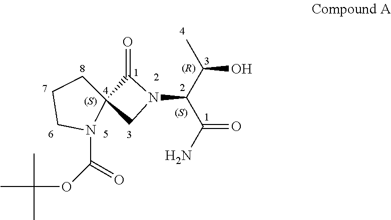

substantially as described herein.

Example embodiment 59: A solid amorphous form of compound A:

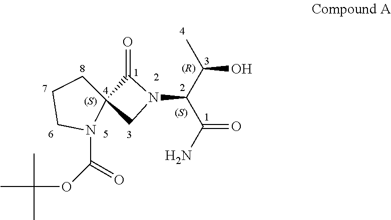

substantially as described herein.

Additional embodiments of each of the aspects will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

### BRIEF DESCRIPTION OF THE DRAWINGS

FIG. 1 shows an experimental X-ray powder diffraction (XRPD) pattern of crystalline Form I of Compound A and an X-ray powder diffraction pattern calculated from a single crystal structure of crystalline Form I of Compound A.

FIG. 2 shows a differential scanning calorimetry (DSC) thermogram of crystalline Form I of Compound A.

FIG. 3 shows an experimental powder X-ray diffraction (XRPD) pattern of crystalline Form II of Compound A and an X-ray powder diffraction pattern calculated from a single crystal structure of crystalline Form II of Compound A.

FIG. 4 shows a Thermogravimetric Analysis (TGA) curve of crystalline Form II of Compound A.

FIG. 5 shows a differential scanning calorimetry (DSC) thermogram of crystalline Form II of Compound A.

FIG. 6 shows desorption profile of water vapor isotherm (DVS) at 25° C. of crystalline Form II of Compound A.

FIG. 7 shows an experimental X-ray powder diffraction (XRPD) pattern of amorphous form of Compound A.

FIG. 8 shows an atomic displacement ellipsoid drawing of crystalline Form I of Compound A based on single crystal X-ray analysis.

FIG. 9 shows a packing diagram of crystalline Form I of Compound A viewed along the a axis.

FIG. 10 shows a molecular conformation drawing of crystalline Form II of Compound A based on single crystal X-ray analysis. Hydrogen atoms are omitted in the figure and only heavy atoms (C, N, O) are displayed.

FIG. 11 shows a packing diagram of crystalline Form II of Compound A viewed along the a axis.

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

As used herein, the term “therapeutically effective amount,” intends an amount of a compound sufficient to show benefit to the individual or subject. This amount prevents, alleviates, abates, or otherwise reduces the severity of a symptom of a disease or disorder responsive to NMDA modulation, such as major depressive disorder.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to an “excipient” includes a single excipient as well as two or more of the same or different excipients, and the like.

The term “about,” particularly in reference to a given quantity, is meant to encompass deviations of plus or minus 5%, 10%, 15% or 20%.

### II. Solid State Forms of Compound A

Described herein are solid state forms of Compound A and methods of preparing them.

The solid state forms can be crystalline (wherein the molecules of the solid form are arranged in a long-range regularly repeating crystal lattice which can be described by a unit cell) or amorphous (wherein the molecules of the solid form are not arranged in any significant regularly repeating manner). Furthermore, and in particular with regards to the crystalline forms, Compound A can be present in the crystal lattice as the only component of the crystal lattice (e.g. Compound A exists as anhydrous form or other non-solvate form in the crystalline solid state). Alternatively, Compound A can be present in the crystal lattice along with another molecule (e.g. water or other solvent molecule) where the other molecule also forms part of the crystal lattice such that overall it exists in a fixed ratio with respect to Compound A (e.g. for water, as a dihydrate of Compound A). Furthermore, a skilled person will also be aware that crystalline forms can often be imperfect in which there may be some vacancies in the crystal lattice and/or there may be some impurities (e.g. molecules other than Compound A or stoichiometric solvent molecules) at some parts of the crystal lattice. However, even in such imperfect forms, the form can still be described as being of a particular crystalline form (e.g. crystalline Form I or crystalline Form II as described herein).

The solid state forms described herein can be identified by any one or more solid state analytical methods. For example, crystalline Form I and/or crystalline Form II of Compound A described herein can be characterized according to any one or more of, e.g., X-ray diffraction (including X-ray powder diffraction), unit cell constants obtained from a single crystal, differential scanning calorimetry, and thermogravimetric analysis.

In some embodiments, the solid state forms described herein can be characterized according to X-ray powder diffraction (XRPD). However, it is known in the art that the intensity and/or measured peaks in the X-ray powder diffractogram of different batches of a solid state form can vary, because of, for example, different experimental conditions and/or preferred orientations. And according to the instrument precision, the measurement error of 2θ value is at ±0.2 2θ. But notwithstanding experimental and machine errors, and principles such as preferred orientation, one skilled in the art can find sufficient information in the XRPD data provided herein to identify crystalline Form I and crystalline Form II without having to rely on all the XRPD data provided.

Accordingly, “substantial similarity” exists between one XRPD pattern and another XRPD pattern when the majority of peaks (such as more than 80% of peaks) in the range of 0 to 40 2θ degrees of the one XRPD can find corresponding peaks in the another XRPD even if corresponding relative intensities of peaks differ.

Unless otherwise indicated, the XRPDs as described herein are obtained using Cu K alpha radiation at 1.54A (λ), 40 kV, and 15 mA.

### A. Crystalline Form I of Compound A

Provided herein is crystalline Form I of Compound A and methods of preparing it.

The crystalline Form I of Compound A appears to be an anhydrous form of Compound A.

In some embodiments, the XRPD of the crystalline Form I as described herein have peaks (2θ) chosen from those having about the following values: 6.9, 8.4, 10.3, and 12.8 each of the diffraction angles being ±0.2 degrees (2θ). In some embodiments, the XRPD of the crystalline Form I as described herein further have one or more peaks (2θ) chosen from those having about the following values: 13.7, 15.3, 15.7, 16.8, 17.3, 18.5, and 19.9 each of the diffraction angles being ±0.2 degrees (2θ).

In some embodiments, the XRPD of the crystalline Form I as described herein can have peaks (2θ) chosen from those having about the following values: 6.9, 8.4, 10.3, 12.8, and 13.7 each of the diffraction angles being ±0.2 degrees (2θ). In some embodiments, the XRPD of the crystalline Form I as described herein can have peaks (2θ) chosen from those having about the following values 6.9, 8.4, 10.3, 12.8, 13.7, 15.3, and 15.7 each of the diffraction angles being ±0.2 degrees (2θ). In some embodiments, the XRPD of the crystalline Form I as described herein can have peaks (2θ) chosen from those having about the following values 6.9, 8.4, 10.3, 12.8, 13.7, 15.3, 15.7, and 16.8 each of the diffraction angles being ±0.2 degrees (2θ). In some embodiments, the XRPD of the crystalline Form I as described herein can have peaks (2θ) chosen from those having about the following values 6.9, 8.4, 10.3, 12.8, 13.7, 15.3, 15.7, 16.8, and 17.3 each of the diffraction angles being ±0.2 degrees (2θ). In some embodiments, the XRPD of the crystalline Form I as described herein can have peaks (2θ) chosen from those having about the following values 6.9, 8.4, 10.3, 12.8, 13.7, 15.3, 15.7, 16.8, 17.3, and 18.5 each of the diffraction angles being ±0.2 degrees (2θ). In some embodiments, the XRPD of the crystalline Form I as described herein can have 3, 4 or 5 peaks (2θ) chosen from those having about the following values 6.9, 8.4, 10.3, 12.8, 13.7, 15.3, 15.7, 16.8, 17.3, 18.5, and 19.9 each of the diffraction angles being ±0.2 degrees (2θ).

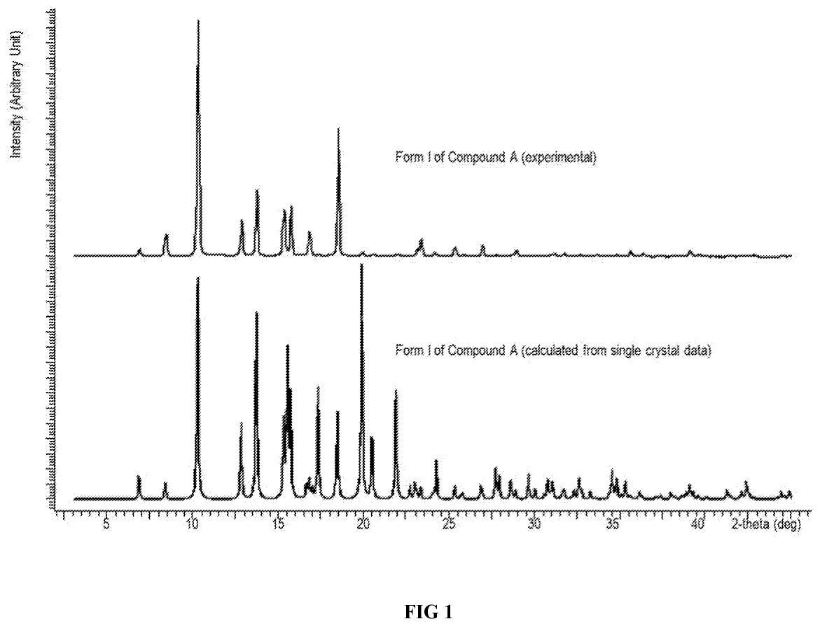

In some embodiments, the crystalline Form I as described herein can have an XRPD substantially similar to one of the XRPDs shown in FIG. 1.

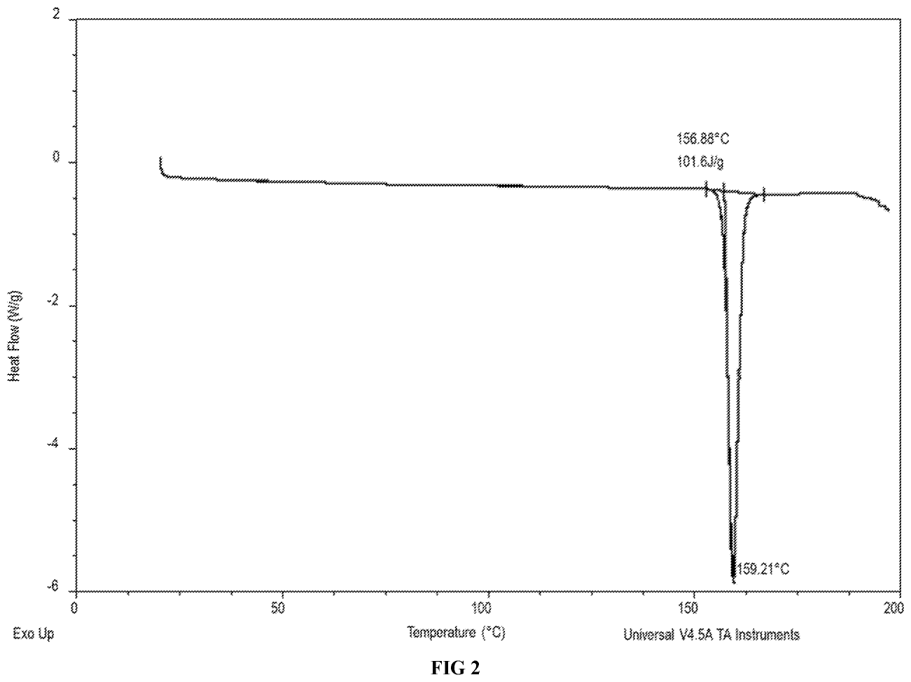

In some embodiments, crystalline Form I of Compound A can be characterized according to a DSC thermogram. For example, provided is an embodiment of the crystalline Form I as described herein having a DSC thermogram substantially similar to that shown in FIG. 2. For example, also provided is an embodiment of the crystalline Form I as described herein having a DSC with endothermic peaks at about 159° C., such as about 159.21° C.

In some embodiments, the crystalline Form I can be present in a solid composition. In some embodiments, the solid composition can be comprised almost entirely of Compound A, though it can contain some additional components (e.g. a solid composition resulting from the synthesis and/or purification of Compound A in which the composition can contain some residual solvent). In such a solid composition, solid Compound A can be present almost entirely as crystalline Form I, or it can be present as a mixture of crystalline Form I with crystalline Form II and/or an amorphous solid form of compound A. The existence and presence of crystalline Form I in a solid composition can be determined by XRPD exhibiting the characteristic 20 peaks for crystalline Form I described herein, as well as other characterization techniques described herein and/or identifiable to a skilled person upon a reading of the present specification.

In some embodiments, a solid composition can comprise crystalline Form I and be substantially free of crystalline Form II and/or of the amorphous form of Compound A. For example, a solid composition comprising crystalline Form I can be at least 99%, at least 95%, at least 90%, or at least 80%, by weight, free of crystalline Form II and/or of the amorphous form of Compound A. Further for example, a solid composition comprising crystalline Form I can be at least 70%, or at least 60%, by weight, free of crystalline Form II and/or of the amorphous form of Compound A. Even further for example, a solid composition comprising crystalline Form I can be at least more than 50% by weight free of crystalline Form II and/or the amorphous form of Compound A. The amount of crystalline Form I relative to crystalline Form II and/or of the amorphous form of Compound A can be determined by methods identifiable to a skilled person, such as, for example, x-ray power diffraction, Raman spectroscopy, solid state nuclear magnetic resonance, differential scanning calorimetry, and dynamic vapor sorption.

In some embodiments, a solid composition comprising crystalline Form I can be substantially free of any other solid forms (crystalline or amorphous) of compound A. For example, a solid composition comprising crystalline Form I can be at least 99%, at least 95%, at least 90%, or at least 80%, by weight, free of any other solid forms of Compound A. Further for example, a solid composition comprising crystalline Form I can be at least 70%, or at least 60%, by weight, free of any other solid forms of Compound A. Even further for example, a solid composition comprising crystalline Form I can be at least more than 50% by weight free of any other solid forms of Compound A. The amount of crystalline Form I relative to other forms of Compound A can be determined by methods identifiable to a skilled person, such as, for example, x-ray power diffraction, Raman spectroscopy, solid state nuclear magnetic resonance, differential scanning calorimetry, and dynamic vapor sorption.

In some embodiments, crystalline Form I of Compound A has a crystal form with orthorhombic crystal system, a P2₁2₁2₁ space group, and the following unit cell dimensions: a=5.85088 (9) Å, b=11.57133 (12) Å, and c=25.8340 (3)Å, α=β=γ=90°, V=1749.02 (4) Å³, Z=4.

Also provided is a method of preparing crystalline Form I of Compound A, comprising dissolving compound A in a first solvent (e.g. ethyl acetate) and heating the solution (e.g. to about 65-70° C.); cooling the solution (e.g. to about 25° C.); and adding a second solvent (e.g. diisopropyl ether) to the solution. In some embodiments, the method further comprises filtration and drying of the collected solid.

Also provided is a method of preparing crystalline Form I of Compound A, comprising heating crystalline Form II of Compound A for dehydration. In some embodiments, heating is conducted at about 80° C.

### B. Crystalline Form II of Compound A

Provided herein is crystalline Form II of Compound A and methods of preparing it.

Crystalline form II of Compound A appears to be a dihydrate form of Compound A.

In some embodiments, the XRPD of the crystalline Form II as described herein has peaks (2θ) chosen from those having about the following values: 9.4, 10.8, 11.9, and 13.0, each of the diffraction angles being ±0.2 degrees (2θ). In some embodiments, the XRPD of the crystalline Form II as described herein further have one or more peaks (2θ) chosen from those having about the following values: 13.7, 15.5, 16.0, 20.0, 20.4, 21.3 and 23.3, each of the diffraction angles being ±0.2 degrees (2θ).

In some embodiments, the XRPD of the crystalline Form II as described herein can have peaks (2θ) chosen from those having about the following values: 9.4, 10.8, 11.9, 13.0, and 13.7 each of the diffraction angles being ±0.2 degrees (2θ). In some embodiments, the XRPD of the crystalline Form II as described herein can have peaks (2θ) chosen from those having about the following values 9.4, 10.8, 11.9, 13.0, 13.7, 15.5, and 16.0 each of the diffraction angles being ±0.2 degrees (2θ). In some embodiments, the XRPD of the crystalline Form II as described herein can have peaks (2θ) chosen from those having about the following values 9.4, 10.8, 11.9, 13.0, 13.7, 15.5, 16.0, 20.0, and 20.4 each of the diffraction angles being ±0.2 degrees (2θ). In some embodiments, the XRPD of the crystalline Form II as described herein can have peaks (2θ) chosen from those having about the following values 9.4, 10.8, 11.9, 13.0, 13.7, 15.5, 16.0, 20.0, 20.4, and 21.3 each of the diffraction angles being ±0.2 degrees (2θ). In some embodiments, the XRPD of the crystalline Form II as described herein can have peaks (2θ) chosen from those having about the following values 9.4, 10.8, 11.9, 13.0, 13.7, 15.5, 16.0, 20.0, 20.4, 21.3 and 23.3 each of the diffraction angles being ±0.2 degrees (2θ). In some embodiments, the XRPD of the crystalline Form II as described herein can have 3, 4 or 5 peaks (2θ) chosen from those having about the following values 9.4, 10.8, 11.9, 13.0, 13.7, 15.5, 16.0, 20.0, 20.4, 21.3 and 23.3 each of the diffraction angles being ±0.2 degrees (2θ).

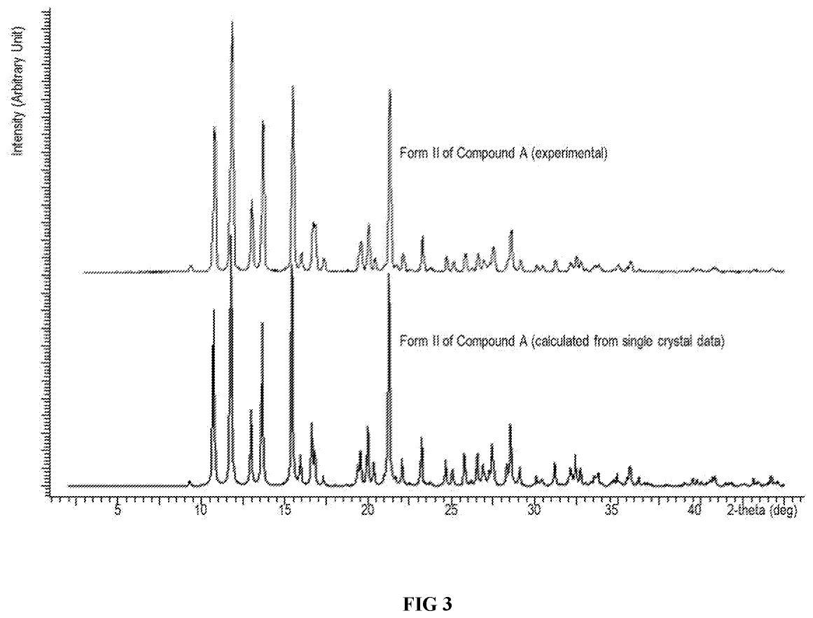

In some embodiments, the crystalline Form II as described herein can have an XRPD substantially similar to one of the two XRPDs shown in FIG. 3.

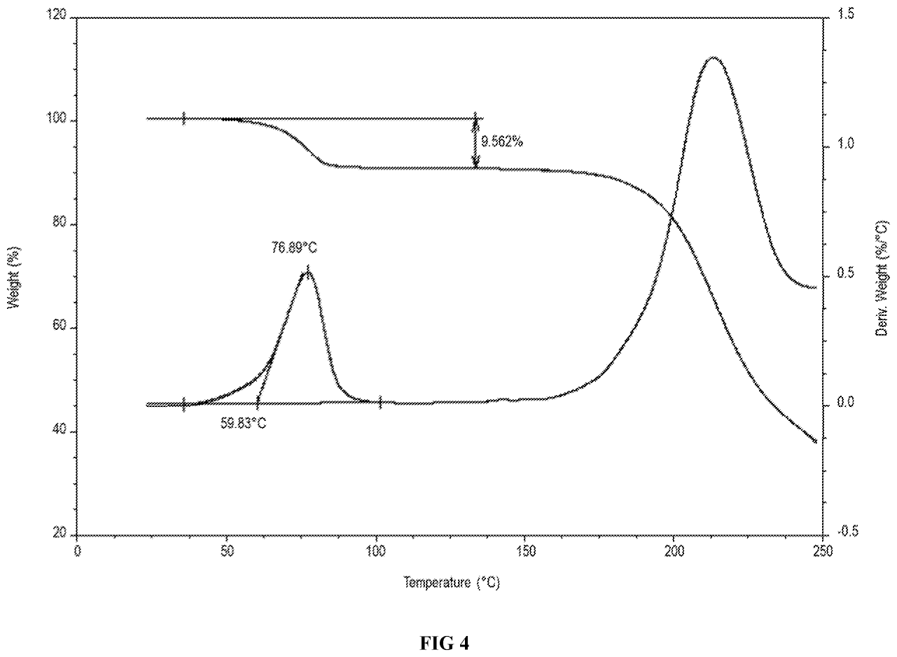

In some embodiments, crystalline Form II of Compound A can be characterized by thermogravimetric analysis (TGA). For example, provided is an embodiment of the crystalline Form II as described herein having a TGA indicating the crystalline Form II as described herein dehydrates approximately at above 60° C., with a loss of water of approximately 9.5% by weight. See, for example, FIG. 4.

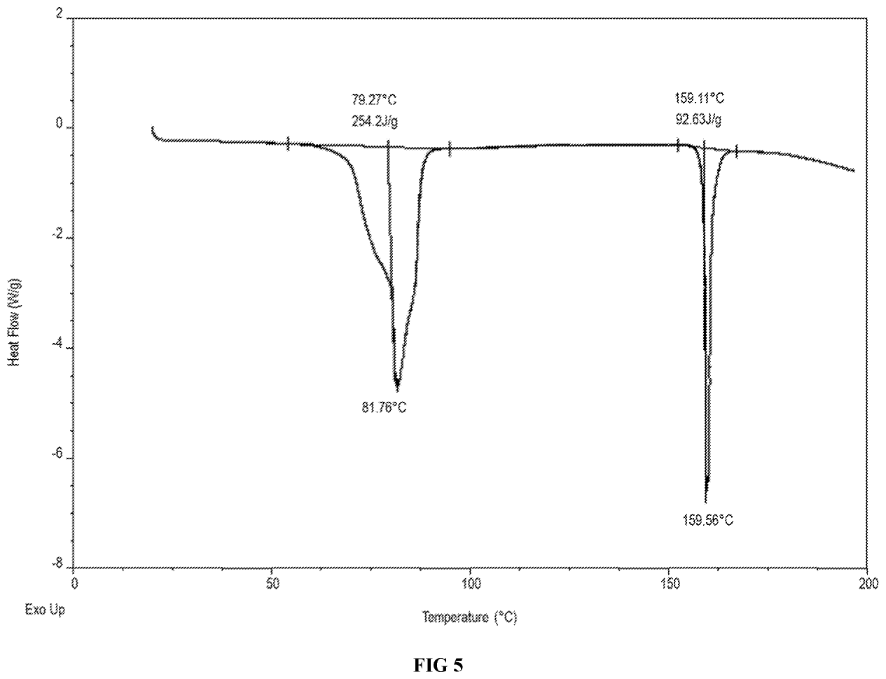

In some embodiments, crystalline Form II of Compound A can be characterized according to a DSC thermogram. The crystalline Form II as described herein having a DSC thermogram indicates that the form II dehydrates at about 82° C. followed by melting at about 159° C. For example, provided is an embodiment of the crystalline Form II as described herein having a DSC thermogram substantially similar to that shown in FIG. 5. Upon dehydration, this form converts into the crystalline Form I, which melts at about 159° C., such as about 159.56° C.

In some embodiments, crystalline Form II of Compound A can be characterized by DVS (Dynamic Vapor Sorption), which indicates that the crystalline Form II loses about 11% water at 0% relative humidity (RH) and did not lose water at or above 20% RH.

In some embodiments, the crystalline Form II can be present in a solid composition. In some embodiments, the solid composition can be comprised almost entirely of Compound A, though it can contain some additional components (e.g. a solid composition resulting from the conversion of a composition comprising crystalline Form I to a composition comprising Form II in which the original composition comprising crystalline Form I had some impurities such as residual solvent). In such a solid composition, solid Compound A can be present almost entirely as crystalline Form II, or it can be present as a mixture of crystalline Form II with crystalline Form I and/or an amorphous solid form of compound A. The existence and presence of crystalline Form II in a solid composition can be determined by XRPD exhibiting the characteristic 20 peaks for crystalline Form I described herein, as well as other characterization techniques described herein and/or identifiable to a skilled person upon a reading of the present specification.

In some embodiments, a solid composition can comprise crystalline Form II and be substantially free of crystalline Form I and/or of the amorphous form of Compound A. For example, a solid composition comprising crystalline Form II can be at least 99%, at least 95%, at least 90%, or at least 80%, by weight, free of crystalline Form I and/or of the amorphous form of Compound A. Further for example, a solid composition comprising crystalline Form II can be at least 70%, or at least 60%, by weight, free of crystalline Form I and/or of the amorphous form of Compound A. Even further for example, a solid composition comprising crystalline Form II can be at least more than 50% by weight free of crystalline Form I and/or of the amorphous form of Compound A. The amount of crystalline Form II relative to crystalline Form I and/or the amorphous form of Compound A can be determined by methods identifiable to a skilled person, such as, for example, x-ray power diffraction, Raman spectroscopy, solid state nuclear magnetic resonance, differential scanning calorimetry, and dynamic vapor sorption.

In some embodiments, a solid composition comprising crystalline Form II can be substantially free of any other solid forms (crystalline or amorphous) of compound A. For example, a solid composition comprising crystalline Form II can be at least 99%, at least 95%, at least 90%, or at least 80%, by weight, free of any other solid forms of Compound A. Further for example, a solid composition comprising crystalline Form II can be at least 70%, or at least 60%, by weight, free of any other solid forms of Compound A. Even further for example, a solid composition comprising crystalline Form II can be at least more than 50% by weight free of any other solid forms of Compound A. The amount of crystalline Form II relative to other forms of Compound A can be determined by methods identifiable to a skilled person, such as, for example, x-ray power diffraction, Raman spectroscopy, solid state nuclear magnetic resonance, differential scanning calorimetry, and dynamic vapor sorption.

In some embodiments, crystalline Form II of Compound A has a crystal form with Orthorhombic crystal system, a P2₁2₁2₁ space group, and the following unit cell dimensions: a=8.9035 (2) Å, b=10.5404 (2) Å, and c=21.3018 (5)Å, V=1999.10 (8) Å³.

Also provided is a method of preparing crystalline Form II of Compound A, comprising mixing such as slurrying crystalline Form I of Compound A with water for some amount of time (e.g. about 4 hours). In some embodiments, the method further comprises filtering and drying the solid. In some embodiments, the amount of slurried Compound A in the water varies from 0.1 to 1.0 g per milliliter of water. In other embodiments, the amount of slurried Compound A in the water varies from 0.1 to 5.0 g per milliliter of water.

### C. Amorphous Form of Compound A

Provided is also an amorphous form of Compound A and methods of preparing it.

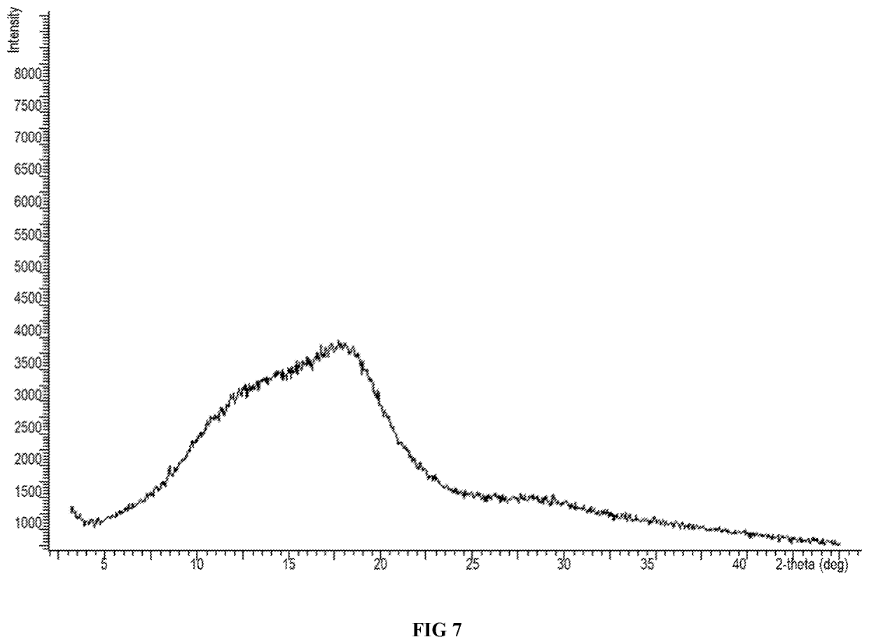

In some embodiments, the amorphous form as described herein can have an XRPD substantially similar to that shown in FIG. 7.

In some embodiments, the amorphous form of Compound A can be present in a solid composition. In some embodiments, the solid composition can be comprised almost entirely of Compound A, though it can contain some additional components (e.g. a solid composition resulting from the spray drying of a solution of Compound A in a solvent that can contain some residual solvent). In such a solid composition, solid Compound A can be present almost entirely as the amorphous form of Compound A, or it can be present as a mixture of the amorphous form of Compound A with crystalline Form I and/or crystalline Form II of compound A. The existence and presence of the amorphous form of Compound A in a solid composition can be determined by XRPD exhibiting the appearance of that in FIG. 7 (i.e. in which there is no indication of crystallinity), as well as other characterization techniques described herein and/or identifiable to a skilled person upon a reading of the present specification.

In some embodiments, a solid composition can comprise the amorphous form of Compound A and be substantially free of crystalline Form I and/or of crystalline Form II of Compound A. For example, a solid composition comprising the amorphous form of Compound A can be at least 99%, at least 95%, at least 90%, or at least 80%, by weight, free of crystalline Form I and/or of crystalline Form II of Compound A. Further for example, a solid composition comprising the amorphous form of Compound A can be at least 70%, or at least 60%, by weight, free of crystalline Form I and/or of crystalline Form II of Compound A. Even further for example, a solid composition comprising the amorphous form of Compound A can be at least more than 50% by weight free of crystalline Form I and/or crystalline Form II of Compound A. The amount of amorphous form relative to crystalline Form I and/or crystalline Form II of Compound A can be determined by methods identifiable to a skilled person, such as, for example, x-ray power diffraction, Raman spectroscopy, solid state nuclear magnetic resonance, differential scanning calorimetry, and dynamic vapor sorption.

In some embodiments, a solid composition comprising crystalline Form I can be substantially free of any other non-amorphous solid forms (e.g. crystalline solid forms) of compound A. For example, a solid composition comprising crystalline Form I can be at least 99%, at least 95%, at least 90%, or at least 80%, by weight, free of any other non-amorphous solid forms of Compound A. Further for example, a solid composition comprising the amorphous form of Compound A can be at least 70%, or at least 60%, by weight, free of any other non-amorphous solid forms of Compound A. Even further for example, a solid composition comprising the amorphous form of Compound A can be at least more than 50% by weight free of any other non-amorphous solid forms of Compound A. The amount of the amorphous form relative to other forms of Compound A can be determined by methods identifiable to a skilled person, such as, for example, x-ray power diffraction, Raman spectroscopy, solid state nuclear magnetic resonance, differential scanning calorimetry, and dynamic vapor sorption.

Provided is also a method of preparing amorphous form of Compound A, comprising drying a solution of Compound A in a solvent. In some embodiments, the solvent is acetone. In some embodiments, the ratio of Compound A to acetone (g/mL) is within the range of 0.05-0.2. In some embodiments, the drying is conducted in the form of spray drying.

### III. Pharmaceutical Compositions and Uses Thereof

Provided is a pharmaceutical composition, comprising crystalline Form I of Compound A and a pharmaceutically acceptable excipient.

In some embodiments, crystalline Form I of Compound A is at least 99%, at least 95%, at least 90%, at least 80%, at least 70%, at least 60%, or at least 50%, by weight, of the total amount of Compound A in the pharmaceutical composition.

Provided is a pharmaceutical composition, comprising crystalline Form II of Compound A and a pharmaceutically acceptable excipient.

In some embodiments, crystalline Form II of Compound A is at least 99%, at least 95%, at least 90%, at least 80%, at least 70%, at least 60%, or at least 50%, by weight, of the total amount of Compound A in the pharmaceutical composition.

Provided is a pharmaceutical composition, comprising amorphous form of Compound A and a pharmaceutically acceptable excipient.

In some embodiments, the amorphous form of Compound A is at least 99%, at least 95%, at least 90%, at least 80%, at least 70%, at least 60%, or at least 50%, by weight, of the total amount of Compound A in the pharmaceutical composition.

Representative excipients should be compatible with the other ingredients of the composition and not harmful for the patient's health. The excipient can be a solid or a liquid or both and can be formulated with Compound A, such as crystalline Form I, crystalline Form II, and/or the amorphous form described herein, as a single dose, for example as a tablet or capsule, which can be prepared from 0.05% to 95% by weight of Compound A described herein. The pharmaceutical compositions described herein can be produced by known pharmaceutical methods, such as those involving mixing the ingredients with pharmaceutically acceptable excipients.

In some embodiments, representative excipients would include but are not limited to: microcrystalline cellulose, lactose, sodium citrate, calcium carbonate, dicalcium phosphate, glycine, disintegrants such as starch, sodium cross-linked carboxymethyl cellulose, composite silicates, and polyethylene glycol with high molecular weight, granulation binders (such as polyvinylpyrrolidone, sucrose, gelatin, and Gum Arabic), and lubricants (such as magnesium stearate, glycerin, and talc).

Also provided is a method of treating a subject in recognized need of treatment for a disease or disorder responsive to NMDA modulation, comprising administering to said subject in need thereof a therapeutically effective amount of a pharmaceutical composition, wherein the pharmaceutical composition comprises a pharmaceutically acceptable carrier and a solid form of Compound A selected from crystalline Form I, crystalline Form II, and amorphous form of Compound A as disclosed herein. The disease or disorder can be a mental disease or disorder, a nervous system disease or disorder, or a neurodegenerative disease or disorder.

In some embodiments, the disease or disorder is selected from autism, anxiety, depression, bipolar disorder, attention deficit disorder, attention deficit hyperactivity disorder (ADHD), schizophrenia, a psychotic disorder, a psychotic symptom, social withdrawal, obsessive-compulsive disorder (OCD), phobia, post-traumatic stress syndrome, a behavior disorder, an impulse control disorder, a substance abuse disorder (e.g., a withdrawal symptom, opiate addiction, nicotine addiction, and ethanol addition), a sleep disorder, a memory disorder (e.g., a deficit, loss, or reduced ability to make new memories), a learning disorder, urinary incontinence, multiple system atrophy, progressive supra-nuclear palsy, Friedrich's ataxia, Down's syndrome, fragile X syndrome, tuberous sclerosis, olivio-ponto-cerebellar atrophy, cerebral palsy, drug-induced optic neuritis, ischemic retinopathy, diabetic retinopathy, glaucoma, dementia, AIDS dementia, Alzheimer's disease, Huntington's chorea, spasticity, myoclonus, muscle spasm, Tourette's syndrome, epilepsy, cerebral ischemia, stroke, a brain tumor, traumatic brain injury, cardiac arrest, myelopathy, spinal cord injury, peripheral neuropathy, acute neuropathic pain, and chronic neuropathic pain.

In some embodiments, the disease or disorder is major depressive disorder.

### IV. Examples

The following examples are illustrative in nature and are in no way intended to be limiting.

Unless otherwise indicated, powder X-ray diffractograms were obtained using Rigaku MiniFlex 600 equipped with the D/tex detector by placing the sample on a zero-background sample holder, with radiation generated from a Cu Kα source at 15 mA and 40 kV, and the instrument was operated over the 2θ range of 3-45° with scan step of 0.020 and scanning speed at 2°/min.

DSC: DSC thermograms were obtained using a TA Instruments DSC Q2000. Approximately 1-2 mg of sample was weighed into a Tzero aluminum pan and hermetically sealed with a Tzero hermetic lid. For crystalline Form II samples, the lid was punched with a pin-hole.
DSC: Sample was scanned from 20 to 180° C. at 10° C./min.

TGA: Approximately 5-10 mg sample was heated from room temperature to 250° C. at 10° C./minute.

DVS: The following method was used for the water vapor desorption analysis using a DVS Advantage (Surface Measurement Systems).
Temperature: 25° C.
RH program: 95, 90, 80, 70, 60, 50, 40, 35, 30, 20, 10, 0%.
Dm/dt (%/min.): 0.0005
Minimum equilibrium time: 120 minutes
Maximum equilibrium time: 2000 minutes

Single crystal analysis of crystalline Form I of Compound A was performed on a Rigaku SuperNova diffractometer, single Cu Kα (λ=1.54184 Å) microfocus source, with Pilatus 200K hybrid pixel array detector at 300 K. Refinements were performed using ShelXL

Single crystal analysis of crystalline Form II of Compound A was performed using Cu Ku radiation (λ=1.54178 Å) on a Bruker AXS D8 Quest CMOS diffractometer equipped with a four axis kappa stage, an I-μ-S microsource X-ray tube with laterally graded multilayer optics, a Photon2 CMOS area detector and an Oxford Cryosystems low temperature device at 150 K.

### Example 1

### Preparation of Form I of Compound A

Crystalline Form I of Compound A was prepared by the following scheme:

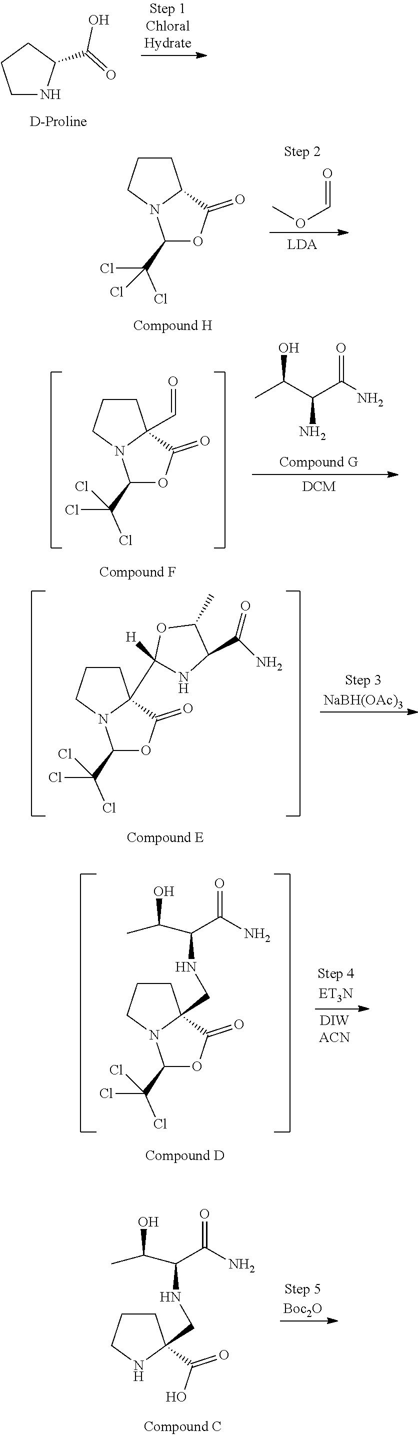

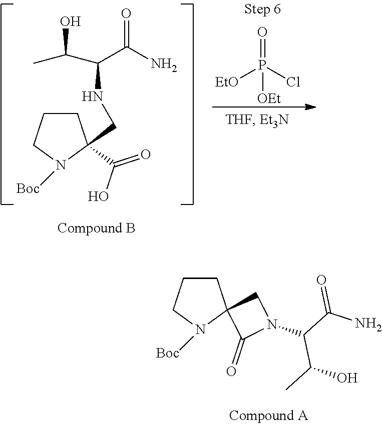

### Step 1: Synthesis of Compound H

A nitrogen purged reactor was charged with acetonitrile, D-proline (69.0 kg), molecular sieves and chloral hydrate (106 kg). The mixture was heated at 50° C. for 5.3 hours. Proton NMR showed complete conversion. The reaction mixture was filtered through a pad of acetonitrile wet Celite and rinsed through with acetonitrile. The filtrate was concentrated to 100 L total volume under vacuum at less than 45° C. N-Butanol (140 L) was added and the mixture was concentrated under vacuum at less than 45° C. for 3.5 hours until no further distillate was observed. The mixture was kept at 20° C. overnight then cooled to 0-5° C. and stirred. The precipitate was collected by pressure filtration, then washed with n-butanol. The resultant solid was dried under vacuum at 45° C. to afford compound H (108.7 kg, 74.2% yield). ¹H-NMR (DMSO-d₆) δ 1.1-1.4 (m, 1H), 1.4-1.7 (m, 1H), 1.7-2.0 (m, 1H), 2.1-2.5 (m, 1H), 3.2-3.4 (m, 1H), 3.5-3.8 (m, 1H), 4.1-4.4 (m, 1H), 5.8 (s, 1H). MS (ESI) m/z (M−H+2H₂O)⁻ 277.94.

### Step 2: Synthesis of Compound F

A nitrogen purged reactor was charged with toluene, MTBE and Compound H (1 eq). The resulting solution was cooled to −55 to −45° C. Lithium diisopropylamide (LDA) solution in THF/n-heptane/ethylbenzene (26.8%, 1.1 eq) was added over 1.3 hour at −50 to −44° C. The resulting solution was stirred at −45±5° C. for 37 minutes then cooled to −75 to −65° C. A solution of methyl formate (2 eq.) in MTBE was added over 45 minutes at less than −60° C. then rinsed in with MTBE. The mixture was stirred for 44 minutes at −70 to −60° C. A second reactor was flushed with nitrogen and charged with deionized water and citric acid monohydrate. The resulting solution was cooled to 0 to 5° C., and the contents of the first reactor was added over 53 minutes at less than 10° C. and rinsed in with MTBE. The mixture was warmed to 11° C. and the phases separated. The aqueous layer was extracted with MTBE then discarded. The main organic layer then the MTBE wash were washed with a solution of sodium chloride (57%) in water (1.8 vol). The combined organics were concentrated under vacuum at less than 50° C. Toluene (2×) was added and the mixture concentrated after each addition until the total volume was 47 L. The mixture was cooled to 35° C. and diluted with methylene chloride affording Compound F as a crude solution with a 65.1% yield. Crystallization of a sample of crude compound F from MTBE/hexanes afforded an analytical sample: ¹H-NMR (DMSO-d₆) δ 1.7-1.8 (m, 1H), 1.8-1.9 (m, 1H), 2.2-2.3 (m, 2H), 3.3-3.4 (m, 1H), 3.5-3.6 (m, 1H), 5.9 (s, 1H), 9.5 (s, 1H). MS (ESI) m/z (M+H)⁺ 272.0.

### Step 3: Synthesis of Compound D

A nitrogen purged reactor was charged with crude compound F solution, methylene chloride and compound G (1.2 eq). The resulting suspension was heated to 30-35° C. for 6 hours then stirred overnight at 20-25° C. to Compound E. An analytical sample of compound E was isolated via silica gel column chromatography (methylene chloride/ethyl acetate eluent) followed by crystallization from ethyl acetate/hexanes. ¹H-NMR (DMSO-d₆) δ 1.2 (d, 3H, J=8 Hz), 1.8-1.9 (m, 2H), 2.0-2.1 (m, 1H), 2.2-2.3 (m, 1H), 3.1-3.2 (m, 1H), 3.2-3.3 (m, 1H), 3.6-3.8 (m, 2H), 4.7 (d, 2H, J=15 Hz), 5.5 (s, 1H), 7.2 (s, 1H), 7.5 (s, 1H). MS (ESI) m/z (M+H)⁺ 372.0.

The crude compound E mixture was cooled to 20° C. and sodium triacetoxyborohydride (3.0 eq) was added over 1.5 H at 20-29° C. then the mixture was stirred 5 hours at 30-35° C. Water was added at 15-20° C. over 49 min with gas evolution. The media was stirred then the phases separated. The aqueous layer was extracted twice with methylene chloride (2×). The combined organics were washed with saturated aqueous sodium bicarbonate. The methylene chloride extracts assayed by HPLC as containing pure compound D with a 78.9% yield. An analytical sample of compound D was crystallized from toluene/hexane and water. ¹H-NMR (DMSO-d₆) δ 1.1 (d, 3H, J=8 Hz), 1.8-1.9 (m, 2H), 2.0-2.1 (m, 2H), 2.7-2.8 (m, 2H), 3.1-3.2 (m, 1H), 3.3-3.4 (m, 1H), 3.6-3.7 (m, 1H), 4.7 (d, 2H, J=6 Hz), 5.6 (s, 1H), 7.0 (s, 1H), 7.1 (s, 1H). MS (ESI) m/z (M+H)⁺ 374.1.

### Step 4: Synthesis of Compound C

The crude solution of Compound D was concentrated under vacuum at less than 45° C. to a total volume of 110 L. Acetonitrile was added and the mixture concentrated to a total volume of 110 L. Acetonitrile, water and triethylamine (6 eq) were added and the mixture heated to 45° C. then stirred f. The mixture was concentrated under vacuum at less than 50° C. to a total volume of less than 110 L. Acetonitrile then Isopropanol were added. The mixture was cooled to 15-20° C., MTBE was added over 1 hour at 15-20° C. and the resultant slurry was stirred at 15-20° C. and the product collected by filtration. The crude solids were slurried in methanol and stirred at 60-65° C. then the suspension was slowly cooled to 20-25° C. The product was collected by filtration and washed with methanol and the solids dried under vacuum at 50° C. to give Compound C with a 72.4% yield. ¹H-NMR (MeOH-d₄) δ 1.23 (3H, d, J=6.4 Hz); 1.9-2.1 (m, 3H), 2.2-2.3 (m, 1H), 2.9 (d, 1H, J=13 Hz), 3.0 (d, 1H, J=6 Hz), 3.1 (d, 1H, J=13 Hz), 3.2-3.3 (m, 1H), 3.4-3.5 (m, 1H), 3.8 (pentet, 1H, J=6 Hz). MS (ESI) m/z (M+H)⁺ 246.2.

### Step 5: Synthesis of Compound B

To a nitrogen purged reactor was charged sequentially acetone, water and Compound C (1 eq). Triethylamine (6 eq) was added to the media in 20 minutes at less than 30° C. and rinsed in with acetone. A solution of di-tert-butyl dicarbonate (1.3 eq) in was added to the mixture at less than 30° C. and rinsed in with acetone (13 L). The mixture was stirred at 20-30° C. n. A solution of di-tert-butyl dicarbonate (0.5 eq) in acetone was added to the mixture. A solution of di-tert-butyl dicarbonate (0.5 eq) in acetone was added. The mixture was concentrated at atmospheric pressure to 65 L total volume. Acetone then THF were added and the mixture concentrated to 65 L total volume. The resulting suspension was cooled to 0-5° C. then the precipitate collected by filtration and washed with THF and dried under vacuum at 45° C. to afford Compound B with a 90.5% yield. ¹H-NMR (DMSO-d₆) δ 1.1 (d, 3H, J=6 Hz), 1.3 (s, 5H), 1.4 (s, 4H), 1.7-1.8 (m, 2H), 1.9-2.0 (m, 1H), 2.2-2.4 (m, 1H), 2.5-3.1 (m, 3H), 3.2-3.5 (m, 3H), 3.6-3.7 (m, 1H), 7.2 (d, 1H, J=16 Hz), 7.4, (d, 1H, J=16 Hz). MS (ESI) m/z (M+H)⁺ 346.3.

### Step 6: Synthesis of Compound A

To a nitrogen purged reactor was charged THF and Compound B (1 eq). Triethylamine (1.8 eq) was added at 20-25° C. and rinsed in with THF. A solution of diethylchlorophosphate (1.8 eq) in THF was added at 20-33° C. After stirring at 25-33° C., a solution of sodium chloride in water was added at 25-30° C. and the phases separated. The aqueous layer was extracted twice with ethyl acetate. The combined organics were concentrated under vacuum at less than 60° C. to a total volume of 65 to 70 L. Ethyl acetate was added and the mixture concentrated to 65 to 70 L total volume. Ethyl acetate followed by a solution of sodium chloride in water (60 L) were added to the mixture. Phosphoric acid was then added to adjust the pH to 2.0. The mixture was stirred at 20-25° C. and the phases separated and the aqueous discarded. The organic layer was washed with a mixture of sodium chloride and aqueous ammonia and the wash back extracted with ethyl acetate. The combined organics were mixed with activated charcoal and stirred overnight then filtered through ethyl acetate wet Celite and rinsed through with ethyl acetate). The filtrate was concentrated under vacuum at less than 60° C. to a total volume of 100 L. Ethyl acetate was added and the mixture concentrated to 100 L after each addition. Ethyl acetate was added and the mixture was cooled to 20-25° C. The mixture was heated to 45-55° C. and the residual solids removed by filtration, washed with ethyl acetate and discarded.

### Step 7: Crystallization of Crystalline Form I of Compound A

The filtrate was concentrated under vacuum at less than 60° C. to a total volume of 105 L. The mixture was heated to 65-70° C. then cooled to 25° C. Diisopropyl ether was added and the mixture stirred at 20-25° C. The precipitate was collected by filtration and washed with diisopropyl ether then dried at 50° C. to give Compound A as a white crystalline powder with 67.2% yield. ¹H-NMR (DMSO-d₆) δ 1.1 (m, 3H), 1.3 (s, 4H), 1.4 (s, 5H), 1.7-1.9 (m, 2H), 2.0-2.3 (m, 2H), 3.1-3.5 (m, 3H), 3.5-4.0 (m, 3H), 4.9 (m, 1H), 7.1-7.6 (m, 2H). MS (ESI) m/z (M+Na)*350.2.

XRPD taken on a sample of Compound A obtained above was essentially the same of that in FIG. 1 (top panel), indicating crystalline Form I of Compound A.

An X-ray powder diffraction pattern calculated from a single crystal structure of crystalline Form I of Compound A is shown in FIG. 1.

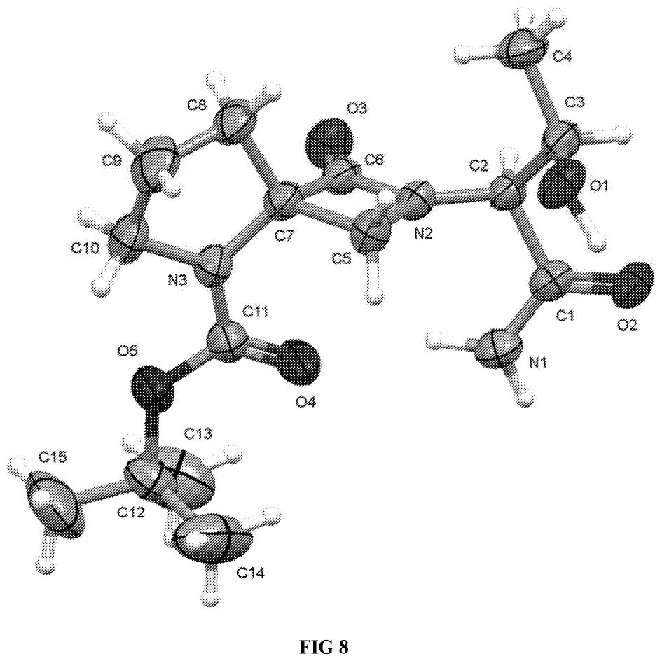

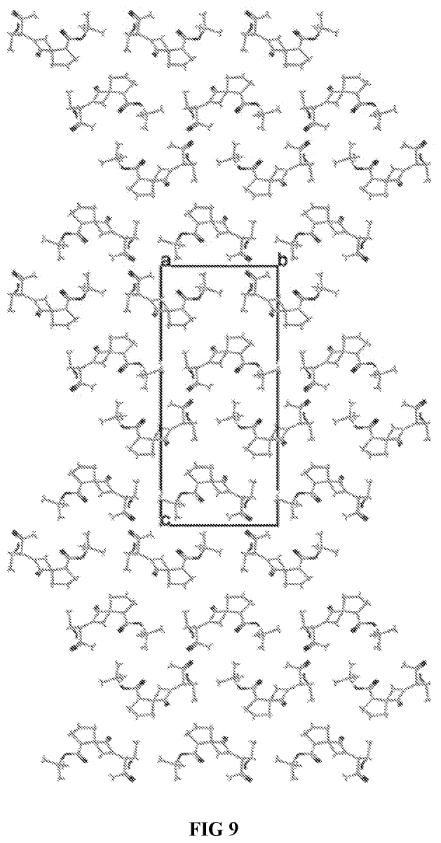

An atomic displacement ellipsoid drawing and packing diagram of crystalline Form I of Compound A based on single crystal X-ray analysis are shown in FIGS. 8-9. Crystalline Form I of Compound A is Orthorhombic crystal system, a P2₁2₁2₁ space group, and the following unit cell dimensions: a=5.85088 (9)Å, b=11.57133 (12)Å, and c=25.8340 (3)Å, α=β=γ=90°, V=1749.02 (4)Å³, Z=4. For Z=4 and a formula weight of 327.38 g/mol, the calculated density is 1.243 g/cm³.

The computer programs used for single crystal analysis and calculated XRPD include ShelXL, CrysAlisPro, Olex2, ShelXT, and Mercury.

### Example 2

### Preparation of Form II of Compound A

In one experiment, about 200 mg of crystalline Form I of Compound A from Example 1 was weighed into a 4 mL scintillation vial and 1 mL of Milli-Q water was added into the vial. The vial was rotated end-to-end at room temperature for 12 days. The residual was filtered with vacuum and air dried at room temperature for 2 days (about 22° C. and 60% RH). The dried solid was ground with mortar and pestle and an XRPD was taken afterwards, which is shown in FIG. 3, top panel.

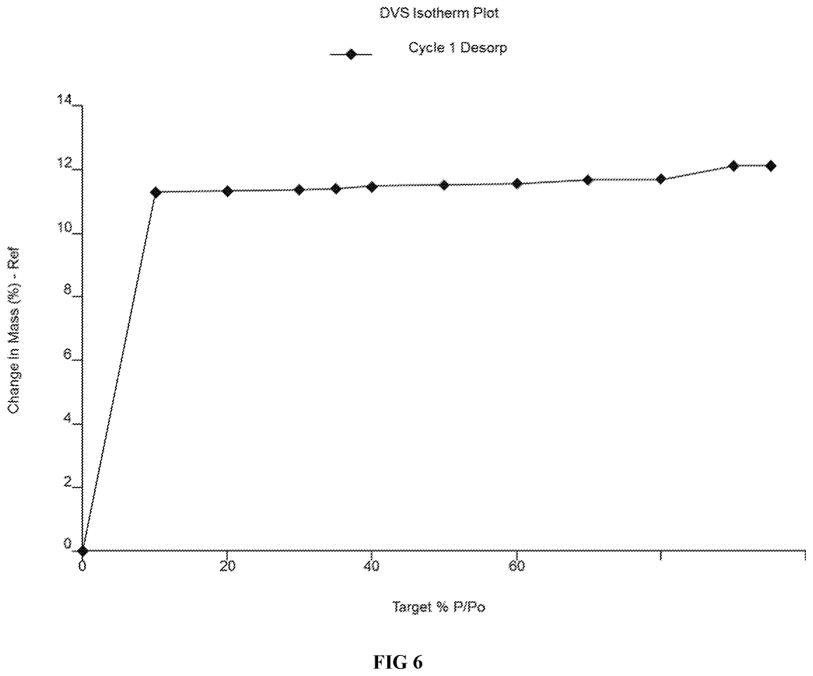

The solid was also subjected to DVS measurement and lost about 11% water at 0% RH. The DVS plot is shown in FIG. 6, which shows crystalline Form II of Compound A did not lose water at or above 20% RH.

In another experiment, about 2 g of crystalline Form I of Compound A from Example 1 was weighed into a 20 mL scintillation vial and 4 mL of Milli-Q water was added into the vial. The resulting suspension was stirred with a spatula and left at room temperature for one day. The residual was then filtered with vacuum and dried in a vacuum oven at room temperature for about 20 hours. XRPD was taken after drying and Form II was confirmed since it was essentially the same as FIG. 3, top panel. A DSC was taken after drying, which is shown in FIG. 5, and showed a possible dehydration event at about 82° C. followed by melting at about 159° C. TGA taken after drying, which is shown in FIG. 4, showed 9.6% weight loss, consistent with that of a dihydrate. The dried sample was then dehydrated in a TGA pan at 80° C. (ramp from room temperature to 80° C. at 10° C./min and then isothermal for 2 min). XRPD and DSC were performed on the dehydrated sample which confirmed that the sample was mostly crystalline Form I with residual amorphous content and very small amount of crystalline Form II.

An X-ray diffraction pattern calculated from a single crystal structure of crystalline Form II of Compound A is shown in FIG. 3, bottom panel.

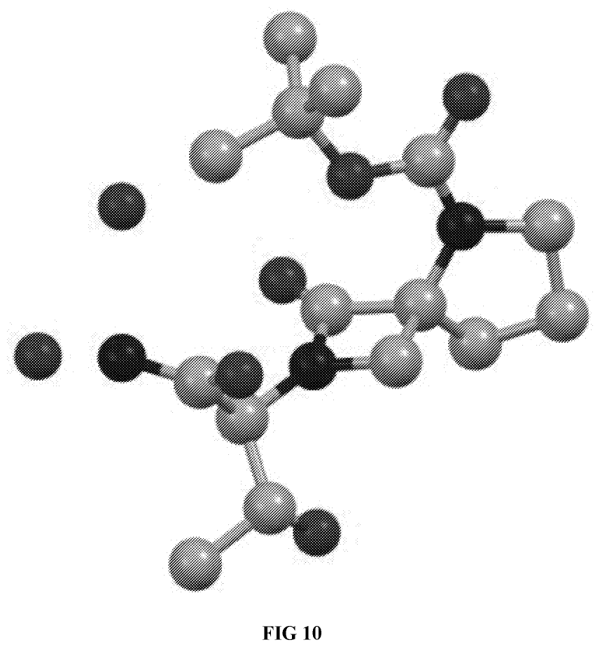

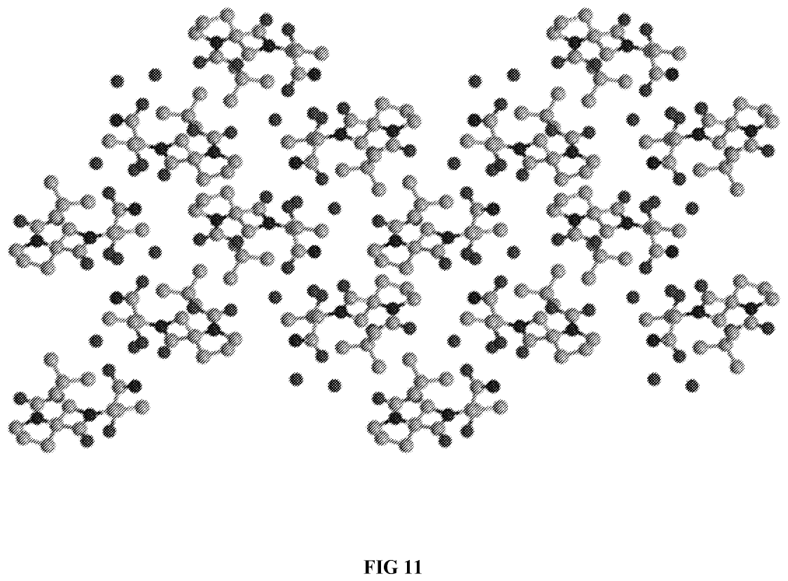

Molecular conformation drawing and packing diagram of crystalline Form II of Compound A based on single crystal X-ray analysis are shown in FIGS. 10-11. Crystalline Form II of Compound A is Orthorhombic crystal system, a P2₁2₁2₁ space group, and the following unit cell dimensions: a=8.9035 (2)Å, b=10.5404 (2)Å, and c=21.3018 (5)Å, α=β=γ=90°, V=1999.10 (8)Å³, Z=4. For Z=4 and a formula weight of 363.41 the calculated density is 1.207 g/cm³.

The computer programs used for single crystal analysis and calculated XRPD include Apex3 v2017.3-0 (Bruker, 2017), SAINT V8.38A (Bruker, 2016), SHELXS97 (Sheldrick, 2008), SHELXL2018/3 (Sheldrick, 2015, 2018), SHELXLE Rev937 (Hübschle et al., 2011).

### Example 3

### Preparation of Amorphous Form of Compound A

One gram of crystalline Form I of Compound A was dissolved in 10 mL acetone. A Büchi mini spray dryer B-290 was employed to spray dry the materials. The input temperature is 65° C. and the output temperature is 44° C. The spray-dried materials were weighed as 0.44 g (44% yield) and analyzed using XRPD. The XRPD is shown in FIG. 7.

### Example 4

### Conversion Between the Crystalline Form I and Crystalline Form II

Competitive slurry in mixtures of water and isopropanol indicates the phase boundary between the crystalline Form I and crystalline Form II is between water activity of 0.66-0.78 at 25° C. At water activity above 0.78, crystalline Form II is the stable form, whereas at water activity below 0.66, crystalline Form I is the stable form

Throughout this specification reference is made to publications such as US and foreign patent applications, journal articles, book chapters, and others. All such publications are expressly incorporated by reference in their entirety, including supplemental/supporting information sections published with the corresponding references, for all purposes unless otherwise indicated.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A method of preparing the solid crystalline Form I of compound A: 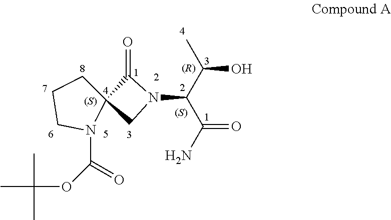
the method comprising:
dissolving compound A in ethyl acetate and heating the solution;
cooling the solution; and
adding a diisopropyl ether to the solution.

2. The method of claim 1, wherein the solution is heated to between about 65° C. and about 70° C.

3. The method of claim 1, wherein the solution is cooled to about 25° C.

4. The method of any one of claims 1 to 3, wherein the solid crystalline Form I of compound A has peaks (2θ) chosen from those having about the following values: 6.9, 8.4, 10.3, and 12.8 in a powder X-ray diffraction pattern.

5. The method of claim 4, wherein the solid crystalline Form I of compound A further has one or more peaks (2θ) chosen from those having about the following values: 13.7, 15.3, 15.7, 16.8, 17.3, 18.5, and 19.9 in a powder X-ray diffraction pattern.

6. The of the method of any one of claims 1 to 3, wherein the solid crystalline Form I of compound A has peaks (2θ) chosen from those having about the following values: 6.9, 8.4, 10.3, 12.8, and 13.7 in a powder X-ray diffraction pattern.

7. The method on any one of claims 1 to 3, wherein the solid crystalline Form I of compound A has peaks (2θ) chosen from those having about the following values 6.9, 8.4, 10.3, 12.8, 13.7, 15.3, and 15.7 in a powder X-ray diffraction pattern.

8. The method of any one of claims 1 to 3, having peaks (2θ) chosen from those having about the following values 6.9, 8.4, 10.3, 12.8, 13.7, 15.3, 15.7, and 16.8 in a powder X-ray diffraction pattern.

9. The method of any one of claims 1 to 3, wherein the solid crystalline Form I of compound A has peaks (2θ) chosen from those having about the following values 6.9, 8.4, 10.3, 12.8, 13.7, 15.3, 15.7, 16.8, and 17.3 in a powder X-ray diffraction pattern.

10. The method of any one of claims 1 to 3, wherein the solid crystalline Form I of compound A has peaks (2θ) chosen from those having about the following values 6.9, 8.4, 10.3, 12.8, 13.7, 15.3, 15.7, 16.8, 17.3, and 18.5 in a powder X-ray diffraction pattern.

11. The method of any one of claims 1 to 3, wherein the solid crystalline Form I of compound A has 3, 4 or 5 peaks (2θ) chosen from those having about the following values 6.9, 8.4, 10.3, 12.8, 13.7, 15.3, 15.7, 16.8, 17.3, 18.5, and 19.9 in a powder X-ray diffraction patterns.

12. The method of any one of claims 1 to 3, wherein the solid crystalline Form I of compound A has an XRPD pattern substantially similar to one of the XRPD patterns shown in FIG. 1.

13. The method of any one of claims 1 to 12, wherein the solid crystalline Form I of compound A has a DSC with endothermic peaks at about 159° C.

14. A solid crystalline form of Compound A: 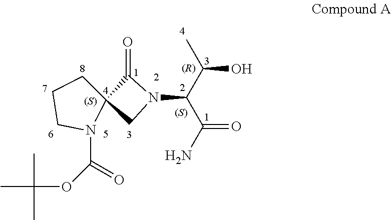
wherein the solid crystalline form is crystalline Form II of Compound A.

15. The solid crystalline form of claim 14, having peaks (2θ) chosen from those having about the following values: 9.4, 10.8, 11.9, and 13.0 in a powder X-ray diffraction patterns.

16. The solid crystalline form of claim 15, further having one or more peaks (2θ) chosen from those having about the following values: 13.7, 15.5, 16.0, 20.0, 20.4, 21.3 and 23.3 in a powder X-ray diffraction pattern.

17. The solid crystalline form of claim 14, having peaks (2θ) chosen from those having about the following values: 9.4, 10.8, 11.9, 13.0, and 13.7 in a powder X-ray diffraction pattern.

18. The solid crystalline form of claim 14, having peaks (2θ) chosen from those having about the following values 9.4, 10.8, 11.9, 13.0, 13.7, 15.5, and 16.0 in a powder X-ray diffraction pattern.

19. The solid crystalline form of claim 14, having peaks (2θ) chosen from those having about the following values 9.4, 10.8, 11.9, 13.0, 13.7, 15.5, 16.0, 20.0, and 20.4 in a powder X-ray diffraction pattern.

20. The solid crystalline form of claim 14, having peaks (2θ) chosen from those having about the following values 9.4, 10.8, 11.9, 13.0, 13.7, 15.5, 16.0, 20.0, 20.4, and 21.3 in a powder X-ray diffraction pattern.

21. The solid crystalline form of claim 14, having peaks (2θ) chosen from those having about the following values 9.4, 10.8, 11.9, 13.0, 13.7, 15.5, 16.0, 20.0, 20.4, 21.3 and 23.3 in a powder X-ray diffraction pattern.

22. The solid crystalline form of claim 14, having 3, 4 or 5 peaks (2θ) chosen from those having about the following values 9.4, 10.8, 11.9, 13.0, 13.7, 15.5, 16.0, 20.0, 20.4, 21.3 and 23.3 in a powder X-ray diffraction pattern.

23. The solid crystalline form of claim 14, having an XRPD pattern substantially similar to one of the two XRPD patterns shown in FIG. 3.

24. The solid crystalline form of any one of claims 14 to 23, having a DSC with endothermic peaks at about 82° C. and at about 159° C.

25. The solid crystalline form of any one of claims 14 to 24, having a TGA showing dehydration approximately at above 60° C., with a loss of water of approximately 9.6% by weight.

26. The solid crystalline form of any one of claims 14 to 25, having a DVS showing about 11% change in mass at 0% RH and 25° C. and the mass does not lose water at or above 20% RH.

27. A solid composition comprising the solid crystalline form of any one of claims 14 to 26, wherein the solid composition is at least 99%, at least 95%, at least 90%, at least 80%, at least 70%, at least 60%, or at least 50%, by weight, free of any other solid forms of Compound A.

28. A pharmaceutical composition comprising the solid crystalline form of any one of claims 14 to 26 and a pharmaceutically acceptable excipient.

29. The pharmaceutical composition of claim 28, wherein the solid crystalline form is at least 99%, at least 95%, at least 90%, at least 80%, at least 70%, at least 60%, or at least 50%, by weight, of the total amount of tert-butyl (S)-2-((2S,3R)-1-amino-3-hydroxy-1-oxobutan-2-yl)-1-oxo-2,5-diazaspiro[3.4]octane-5-carboxylate in the pharmaceutical composition.

30. A solid amorphous form of Compound A:

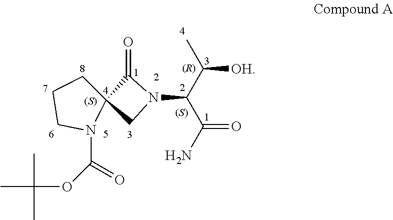

31. The solid amorphous form of claim 30, having an amorphous halo in a powder X-ray diffraction pattern.

32. The solid amorphous form of claim 30, having an XRPD pattern that is substantially similar to FIG. 7.

33. A pharmaceutical composition comprising the amorphous form of any one of claims 30 to 32 and a pharmaceutically acceptable excipient.

34. The pharmaceutical composition of claim 33, wherein the amorphous form is at least 99%, at least 95%, at least 90%, at least 80%, at least 70%, at least 60%, or at least 50%, by weight, of the total amount of tert-butyl (S)-2-((2S,3R)-1-amino-3-hydroxy-1-oxobutan-2-yl)-1-oxo-2,5-diazaspiro[3.4]octane-5-carboxylate in the pharmaceutical composition.

35. A method of treating a subject in recognized need of treatment for a disease or disorder responsive to NMDA modulation, comprising administering to said subject in need thereof a therapeutically effective amount of a pharmaceutical composition of any one of claims 28, 29, 33, and 34.

36. The method of claim 35, wherein the disease or disorder is selected from autism, anxiety, depression, bipolar disorder, attention deficit disorder, attention deficit hyperactivity disorder (ADHD), schizophrenia, a psychotic disorder, a psychotic symptom, social withdrawal, obsessive-compulsive disorder (OCD), phobia, post-traumatic stress syndrome, a behavior disorder, an impulse control disorder, a substance abuse disorder, a sleep disorder, a memory disorder, a learning disorder, urinary incontinence, multiple system atrophy, progressive supra-nuclear palsy, Friedrich's ataxia, Down's syndrome, fragile X syndrome, tuberous sclerosis, olivio-ponto-cerebellar atrophy, cerebral palsy, drug-induced optic neuritis, ischemic retinopathy, diabetic retinopathy, glaucoma, dementia, AIDS dementia, Alzheimer's disease, Huntington's chorea, spasticity, myoclonus, muscle spasm, Tourette's syndrome, epilepsy, cerebral ischemia, stroke, a brain tumor, traumatic brain injury, cardiac arrest, myelopathy, spinal cord injury, peripheral neuropathy, acute neuropathic pain, and chronic neuropathic pain.

37. The method of claim 36, wherein the substance abuse disorder is elected from a withdrawal symptom, opiate addiction, nicotine addiction, and ethanol addition.

38. The method of claim 36, wherein the memory disorder is selected from a deficit, loss, and reduced ability to make new memories.

39. The method of claim 35, wherein the disease or disorder is major depressive disorder.

40. A crystal form of tert-butyl (S)-2-((2S,3R)-1-amino-3-hydroxy-1-oxobutan-2-yl)-1-oxo-2,5-diazaspiro[3.4]octane-5-carboxylate dihydrate having an Orthorhombic crystal system, a P2₁2₁2₁ space group, and the following unit cell dimensions: a=8.9035 (2)Å, b=10.5404 (2)Å, and c=21.3018 (5)Å, α=β=γ=90°, V=1999.10 (8)Å³, Z=4.

41. A solid crystalline Form II of compound A: 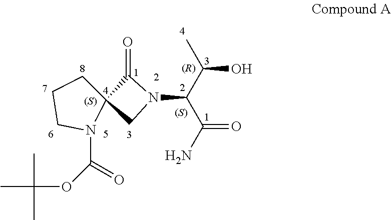
substantially as described herein.

42. A solid amorphous form of compound A: 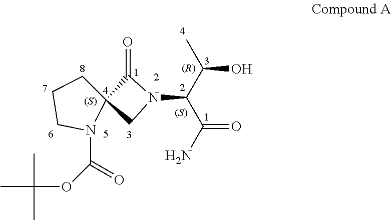
substantially as described herein.
